(19) United States
(12) Patent Application Publication
(10) Pub. No.: US 2016/0128579 A1
(43) Pub. Date: May 12, 2016

(54) PROBE AND SUBJECT INFORMATION ACQUIRING APPARATUS

(71) Applicant: CANON KABUSHIKI KAISHA, Tokyo (JP)

(72) Inventor: Atsushi Kandori, Ebina-shi (JP)

(21) Appl. No.: 14/936,478

(22) Filed: Nov. 9, 2015

(30) Foreign Application Priority Data
Nov. 12, 2014 (JP) 2014-229606

(51) Int. Cl. A61B 5/00

(52) U.S. Cl. CPC A61B 5/0095; A61B 2562/0204

(57) ABSTRACT

A probe includes a plurality of element groups, the element group including a first element and a plurality of second elements which are arranged in a first direction, the plurality of element groups being arranged in a second direction intersecting with the first direction. An acoustic lens having a curvature in the first direction is arranged on the first element. When transmission and reception based on a pulse echo technique are performed, the transmission and reception are performed with respect to the subject by using only the first element, and when reception of a photoacoustic wave is performed, the reception from the subject is performed by using the entire element group constituted by the first element and the plurality of second elements.

### BACKGROUND OF THE INVENTION

1. Field of the Invention

The present invention relates to a probe that can perform transmission and reception of an acoustic wave such as an ultrasonic wave and reception of a photoacoustic wave by way of a photoacoustic effect, a subject information acquiring apparatus using the probe, and the like. Hereinafter, the acoustic wave is used as a term that includes a sonic wave, an ultrasonic wave, a photoacoustic wave, and the like but may be represented by an ultrasonic wave in some cases.

2. Description of the Related Art

When a subject is subjected to an ultrasonic wave (transmission of the ultrasonic wave), and a reflected wave of the ultrasonic wave at a region having a different acoustic impedance in the subject is detected (reception of the ultrasonic wave), it is possible to observe an internal part of the subject. This is referred to as a pulse echo technique. When a subject is subjected to pulsed light, and an ultrasonic wave generated by a photoacoustic effect is received, it is possible to detect (receive) distributions, concentrations, and the like of glucose and hemoglobin contained in blood in the subject. This is referred to as a photoacoustic imaging technique (see US Patent Laid-Open No. 2007/0287912). A transducer called a search unit that can interconvert an electric signal and vibration can be used as a unit configured to perform transmission and reception of the ultrasonic wave by using the pulse echo technique and the reception of the photoacoustic wave by using the photoacoustic imaging technique. An apparatus that is provided with the search unit to be connected to an apparatus main body and configured to perform the transmission and reception of an ultrasonic wave signal is referred to as a probe.

FIG. 9A, FIG. 9B, and FIG. 9C are schematic views of an example probe. These diagrams illustrate a probe 900, a casing 901, a search unit 902, an acoustic lens 903, a cable 904, and a connector 905. As illustrated in the external appearance schematic view of FIG. 9A, the acoustic lens 903 is arranged on a surface of the search unit 902 (see FIG. 9C) configured to perform input and output of the ultrasonic wave, and the acoustic lens 903 and the search unit 902 are enclosed by the casing 901. The search unit 902 is used while being electrically connected to an external apparatus outside the probe via the cable 904 and the connector 905. The search unit 902 is a chip constituted by elements 911 and a member 920 that holds the elements 911. It is noted that an X direction in the drawings is referred to as the elevation direction, and a Y direction is referred to as the azimuth direction in the present specification.

FIG. 9B illustrates an arrangement of the elements 911 provided to the search unit 902 as seen from the acoustic lens 903 side. The plurality of rectangular elements 911 are arranged along the azimuth direction (Y direction). As illustrated in FIG. 9C corresponding to the IXC-IXC cross section of FIG. 9B, the acoustic lens 903 is arranged via an adhesive layer 910 on the element 911 provided to the search unit 902. A cable obtained by binding together a plurality of fine-wire coaxial cables is used as the cable 904, and an electric connection to the external apparatus can be established for each of the elements 911. When the element 911 is applied with a pulsed voltage, the ultrasonic wave can be output from the element 911 (transmission of the ultrasonic wave). In addition, when a signal output from the element 911 that has received the ultrasonic wave is extracted, the ultrasonic wave can be detected (reception of the ultrasonic wave).

Since the acoustic lens 903 has a curvature, when the elements 911 are driven in the same phase, a transmitted sound pressure is increased at a predetermined distance from the surface of the acoustic lens 903. Similarly, when the ultrasonic wave reflected by a reflector arranged at the predetermined distance is input to the acoustic lens 903, the output from the element 911 is maximized. With the existence of the acoustic lens 903, a slice resolution (resolution in the X direction in FIG. 9C) of the reflector within a predetermined distance range from the lens in a Z direction is increased, and a resolution for identifying an object is increased. It is noted that, in the following explanation, the acoustic wave transmitted and received by using the normal pulse echo technique is referred to as an ultrasonic wave, which is representative thereof, and the acoustic wave generated by the photoacoustic imaging technique may be referred to as a photoacoustic wave in some cases.

In general, specifications of optimal search units are different from each other in transmission and reception using the pulse echo technique and the reception using the photoacoustic imaging technique. For this reason, in a case where the transmission and reception using the pulse echo technique and the reception using the photoacoustic imaging technique are performed by the same search unit, the spatial resolution (slice resolution) that can be acquired by the search unit is lower than in a case where the signals are acquired by respectively appropriate different search units. On the other hand, in a case where the search unit for the transmission and reception using the pulse echo technique and the search unit for the reception of the photoacoustic imaging technique are respectively constituted by different search units, it is possible to use the search units respectively having the optimal specifications, and a decrease in the slice resolution hardly occurs. However, the volume occupied by the other search units in total may be markedly increased, or an image positional deviation caused by the other search units may occur in some cases.

### SUMMARY OF THE INVENTION

The present invention provides an apparatus such as a probe that can be used for both transmission and reception using the pulse echo technique and reception using the photoacoustic imaging technique.

A probe according to an aspect of the present invention has the following characteristic. That is, the probe includes a plurality of element groups, the element group including a first element and a plurality of second elements which are arranged in a first direction, the plurality of element groups being arranged in a second direction intersecting with the first direction. An acoustic lens having a curvature in the first direction is arranged on the first element. When transmission and reception using a method of transmitting an acoustic wave to a subject and receiving a reflected wave from a subject are performed, the transmission and reception are performed with respect to the subject by using only the first element. When reception of a photoacoustic wave is performed, the reception from the subject is performed by using the entire element group constituted by the first element and the plurality of second elements.

Further features of the present invention will become apparent from the following description of exemplary embodiments with reference to the attached drawings.

### BRIEF DESCRIPTION OF THE DRAWINGS

FIG. 1A is a schematic view for describing a probe according to a first exemplary embodiment.

FIG. 1B is a perspective view for describing the probe according to the first exemplary embodiment.

FIG. 1C is a cross-sectional view for describing the probe according to the first exemplary embodiment.

FIG. 2 is an explanatory diagram for describing an internal part of the probe according to the first exemplary embodiment.

FIG. 3A is an explanatory diagram for describing the internal part of the probe according to a second exemplary embodiment.

FIG. 3B is an explanatory diagram for describing another mode example of the internal part of the probe according to the second exemplary embodiment.

FIG. 4A is an explanatory diagram for describing the internal part of the probe according to a third exemplary embodiment.

FIG. 4B is an explanatory diagram for describing another mode example of the internal part of the probe according to the third exemplary embodiment

FIG. 5A is an explanatory diagram for describing the internal part of the probe according to a fourth exemplary embodiment.

FIG. 5B is an explanatory diagram for describing another mode example of the internal part of the probe according to the fourth exemplary embodiment.

FIG. 6A is an explanatory diagram for describing a transducer of the probe according to a fifth exemplary embodiment.

FIG. 6B is a front view for describing lines of elements of the probe according to the fifth exemplary embodiment.

FIG. 6C is a back-side view for describing the lines of the elements of the probe according to the fifth exemplary embodiment.

FIG. 6D is a cross-sectional view for describing the lines of the elements of the probe according to the fifth exemplary embodiment.

FIG. 7 is an explanatory diagram for describing an information acquiring apparatus according to a sixth exemplary embodiment.

FIG. 8 is an explanatory diagram for describing the internal part of the probe according to a seventh exemplary embodiment.

FIG. 9A is a schematic view for describing an ultrasonic wave probe in a related art.

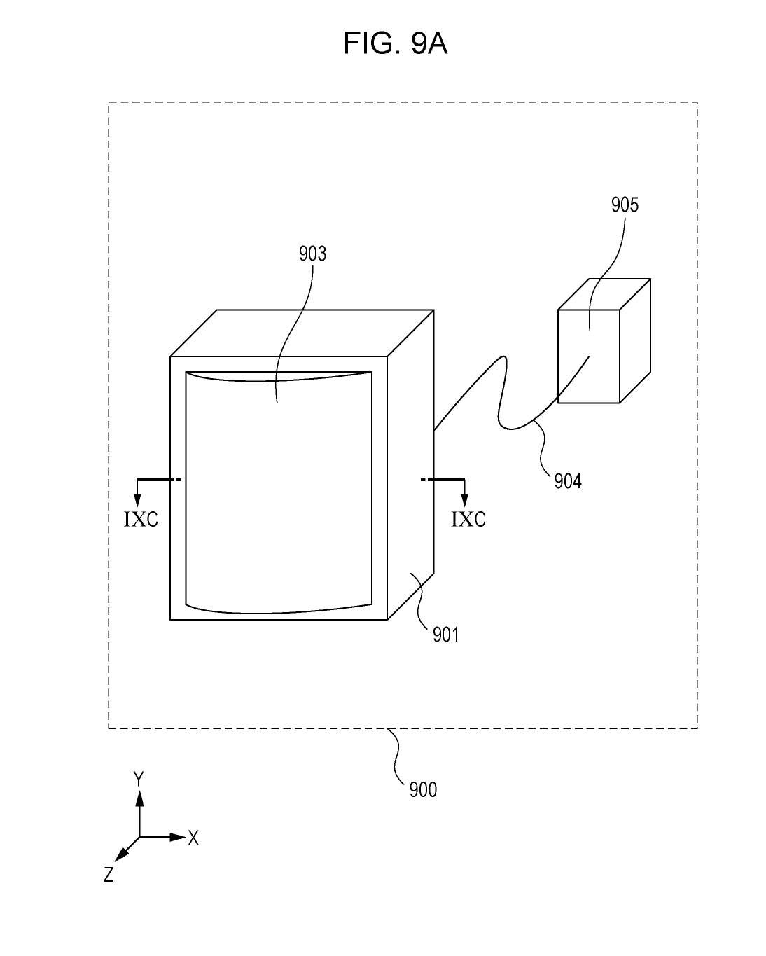

FIG. 9B is a perspective view for describing the ultrasonic wave probe in the related art.

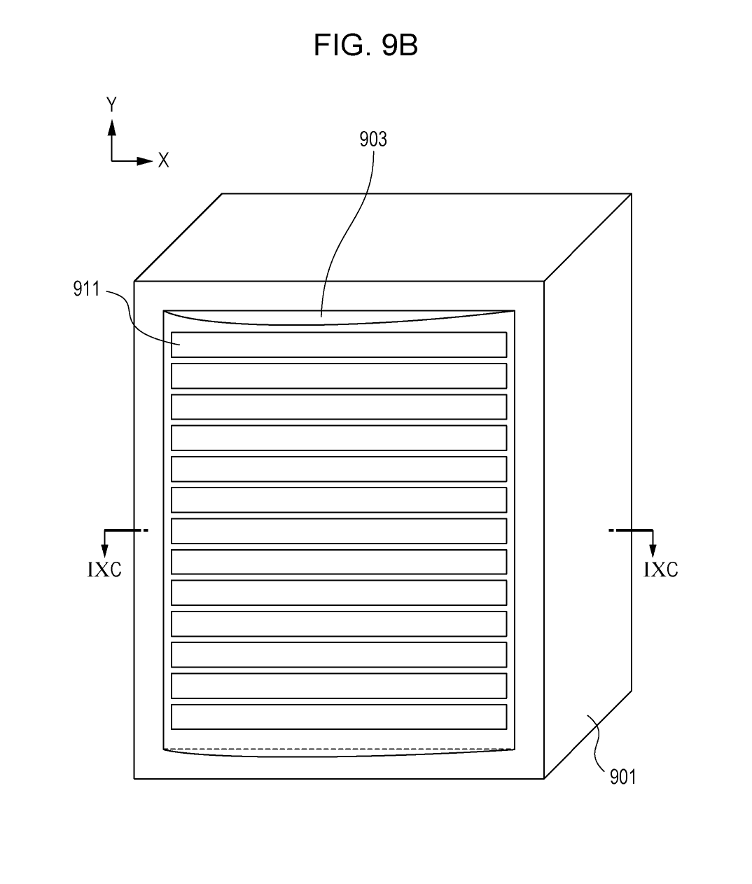

FIG. 9C is a cross-sectional view for describing the ultrasonic wave probe in the related art.

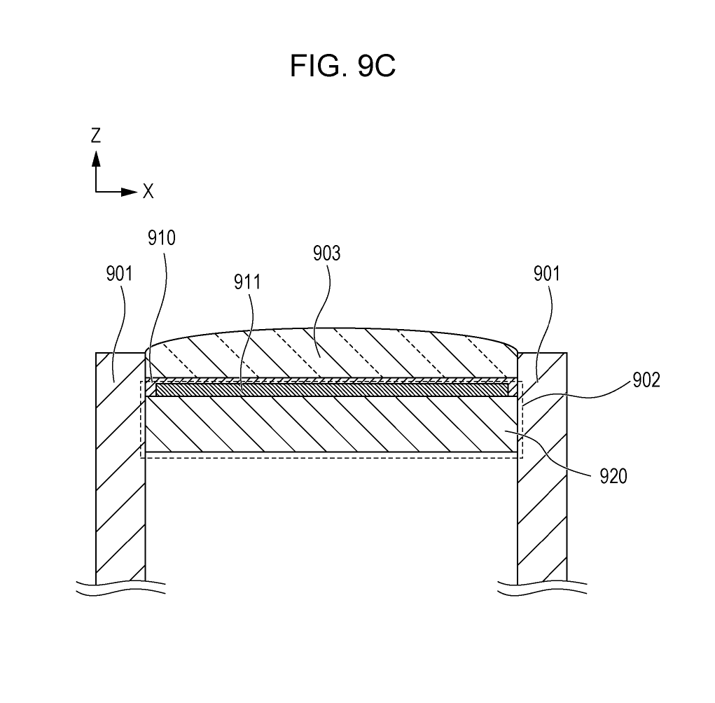

### DESCRIPTION OF THE EMBODIMENTS

According to exemplary embodiments of the present invention, for example, a plurality of elements (element group) are arranged in a first direction (elevation direction) of a probe, and an acoustic lens having a curvature in the first direction is arranged only on a first element (typically, a single first element) at a central part. Herein, in transmission and reception of an ultrasonic wave, it is possible to perform transmission and reception via the acoustic lens by using the first element arranged below the acoustic lens. On the other hand, in reception of a photoacoustic wave, a signal obtained by performing delay and sum processing (phases of the plurality of reception signals are adjusted and added) on reception signals from the plurality of second elements arranged in a region where the acoustic lens does not exist is added to an output of the element arranged below the acoustic lens. A plurality of element groups are arranged in a direction intersecting with the first direction (typically a direction orthogonal to the first direction). A portion that is made of the same material as the acoustic lens and has no curvature in the first direction is arranged on the plurality of second elements, for example. According to the above-described configuration, only the first element can be used at the time of the transmission and reception performed using the pulse echo technique, and at the time of the reception of the photoacoustic wave, the entire element group in the first direction which is constituted by the first element and the plurality of second elements can be used.

The acoustic lens is, for example, a cylindrical lens as described above, but lenses having other modes such as a cylindrical lens constituted by a Fresnel lens and a cylindrical lens having a slight curvature also in a long-side direction can also be used. The acoustic lens is typically arranged at the central part but can also be arranged at an end part depending on the situation. In a case where the acoustic lens is arranged at the end part, the element to be arranged at a position corresponding to this end part is not divided, and the element at the central part needs to be divided. In a case where a sound axis inclined with respect to the probe (sound axis inclined from the Z axis corresponding to a direction perpendicular to the XY plane) is prepared, for example, only two regions on the left and right sides including the region where the acoustic lens exists and the region where the acoustic lens does not exist are used. A plurality of regions including the region where the acoustic lens exists and the region where the acoustic lens does not exist are arranged so as to be asymmetrical to each other with a central line therebetween. Each of the elements has at least one cell or vibrator, but in terms of the configurations thereof, the structures of the cells, the arrangement densities of the cells, the numbers, the arrangement patterns, and the like may be the same or different from one another. A case is conceivable where a portion made of the same material as the acoustic lens is not arranged on the second element, but in that case, any protective film may be provided in view of safety. The portion made of the same material as the acoustic lens can have a curvature different from that of the acoustic lens.

That is, the above-described modifications and alterations may be sufficient when the degradation in the characteristics of the detection for the acoustic wave and the photoacoustic wave by using the pulse echo technique and the photoacoustic imaging technique is small, and also both slice resolutions can be set to be close to each other. Hereinafter, exemplary embodiments of the present invention will be described. However, the present invention is not limited to these exemplary embodiments, and various modifications and alterations within the gist of the present invention can be made.

Hereinafter, the probe and the like according to the exemplary embodiments of the present invention will be described in detail by using the drawings.

### First Exemplary Embodiment

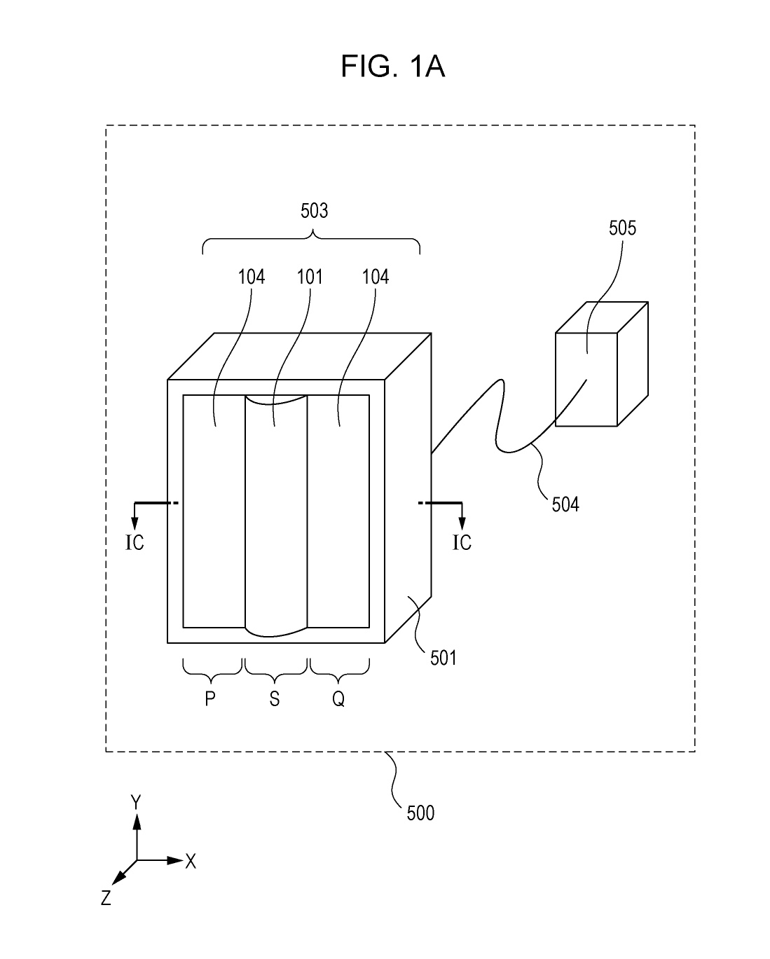

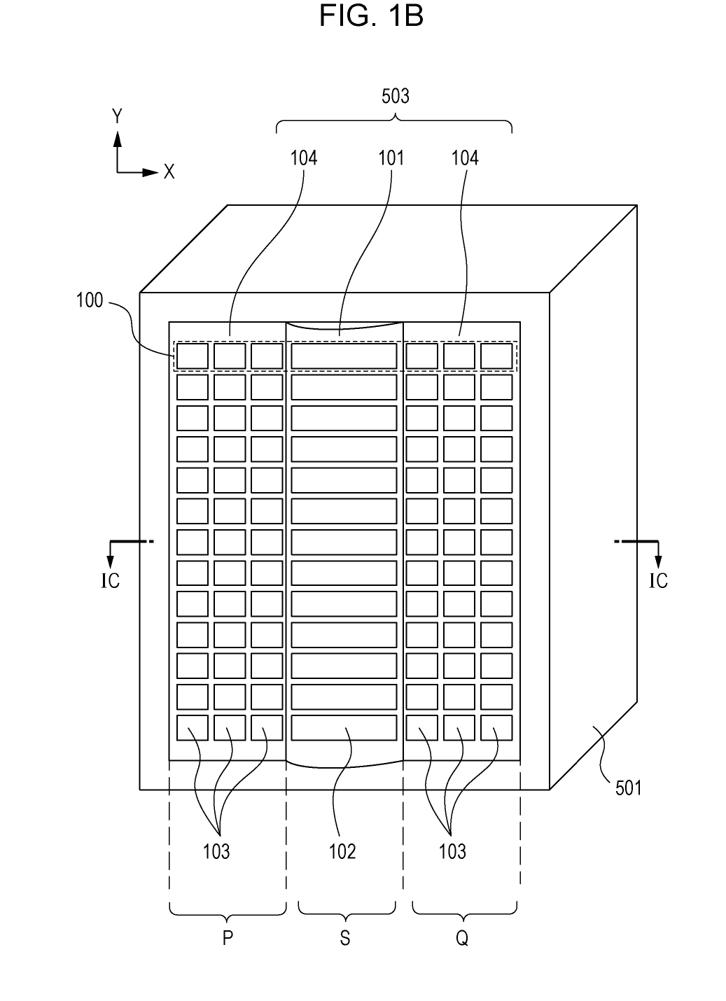

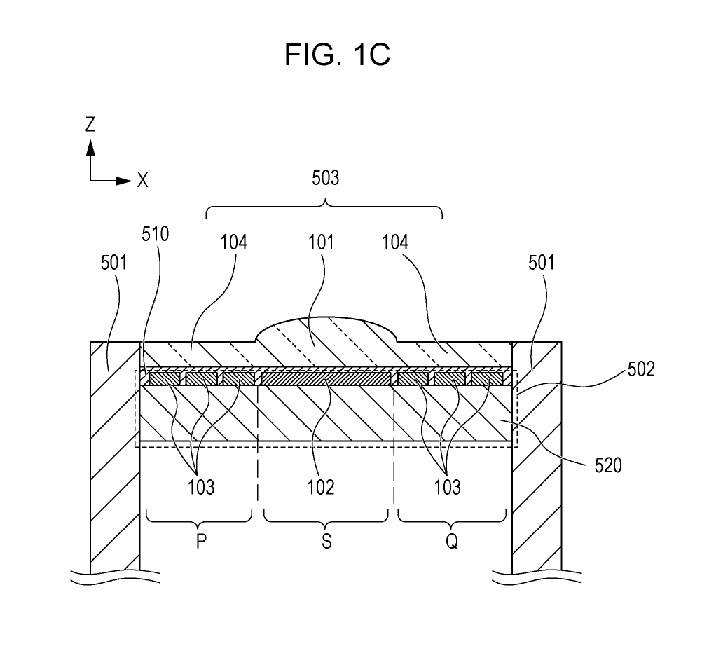

FIG. 1A, FIG. 1B, and FIG. 1C illustrate a probe according to the present exemplary embodiment. These drawings illustrate an element group 100 including a plurality of elements arranged in an elevation direction (X direction, first direction), an acoustic lens 101, a first element 102, a second element 103, and resin 104. These drawings also illustrate a probe 500, a casing 501, a search unit 502, a transmission and reception surface 503 for the ultrasonic wave and the photoacoustic wave, a cable 504, a connector 505, and an adhesive layer 510. The search unit 502 is a chip constituted by the first element 102, the second element 103, and a member 520 that holds the first element 102 and the second element 103.

In FIG. 1A corresponding to the external appearance schematic view of the probe according to the present exemplary embodiment, the surface of the search unit 502 of the probe 500 is divided into three rectangular regions P, S, and Q extending in a Y direction. The acoustic lens 101 having a curvature in the X direction is arranged in the region S corresponding to a central part, and the acoustic lens is not arranged in the regions P and Q corresponding to parts at sides. Herein, the resin 104 made of the same material as the acoustic lens 101 and provided with a flat surface is arranged in the regions P and Q as a protective layer. The transmission and reception surface 503 constituted by the acoustic lens 101 and the resin 104 is enclosed by the casing 501. As illustrated in the cross-sectional view of FIG. 1C, the search unit 502 is arranged under the acoustic lens 101 and the resin 104 via the adhesive layer 510.

As described above, except for special cases, the Z axis corresponding to a direction perpendicular to the XY plane is referred to as the sound axis. The shortest interval at which a plurality of reflectors arranged in the X direction can be detected is referred to as the slice resolution, and the shortest interval at which a plurality of reflectors arranged in the Y direction can be detected is referred to as the azimuth resolution. The X direction corresponds to the elevation direction, and the Y direction corresponds to the azimuth direction. According to the present exemplary embodiment, the elevation direction is orthogonal to the azimuth direction.

FIG. 1B illustrates the arrangement of the first element 102 and the second element 103 provided to the probe 500 as seen from the transmission and reception surface 503 side. FIG. 1C illustrates the IC-IC cross section of FIG. 1B. In the search unit 502, the single first element 102 and the plurality of second elements 103 are aligned in line in the X direction. The first element 102 and the plurality of second elements 103 aligned in line in the elevation direction are collectively referred to as the element group 100 in the elevation direction. Herein, descriptions will be given while m sets of the element groups 100 in the elevation direction are arranged along the azimuth direction. The present invention including the present exemplary embodiment is characterized in that the transmission and reception using the pulse echo technique are performed by using the first element 102, and the reception using the photoacoustic imaging technique is performed by using the first element 102 and the plurality of second elements 103 all included in the element group 100 in the elevation direction.

In FIG. 1C, the first element 102 is arranged, via the adhesive layer 510, below the acoustic lens 101 arranged in the region S. A width of the first element 102 in the X direction is substantially the same as the area of the acoustic lens 101 having the curvature in the X direction. On the other hand, the resin 104 having no curvature is arranged in the regions P and Q, and the plurality of second elements 103 are arranged below the resin 104 via the adhesive layer 510. Herein, n pieces of the second elements 103 exist in the single element group 100 in the descriptions.

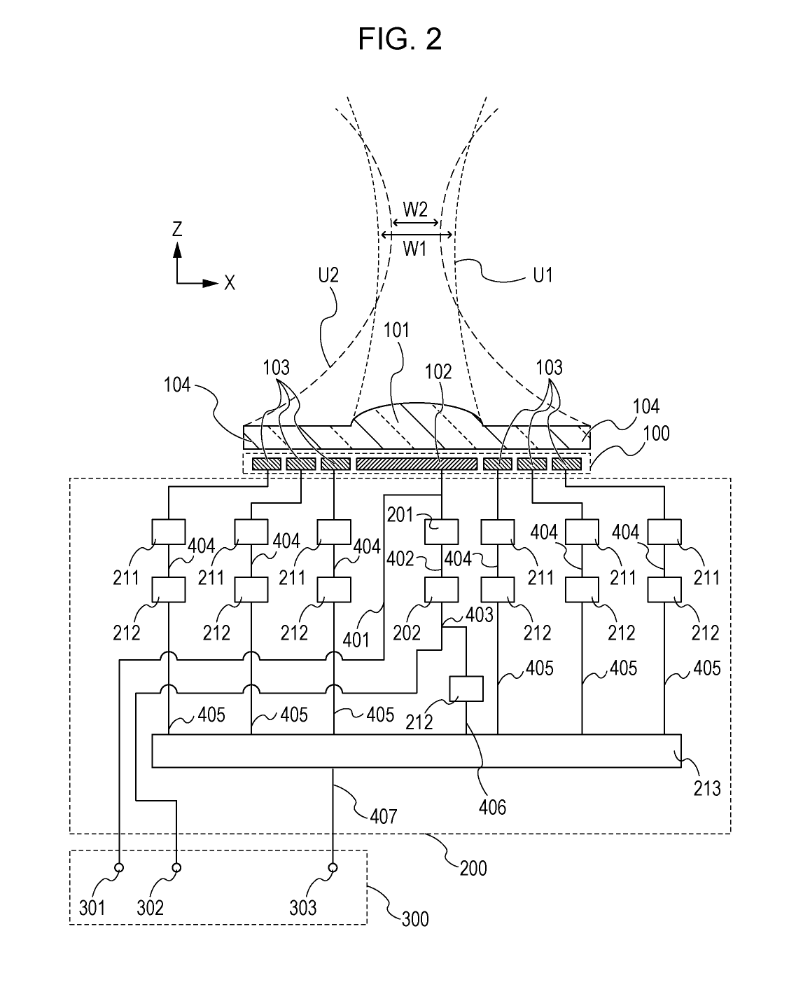

FIG. 2 is a schematic view for describing the element, the circuit, the terminal, and the like in the probe 500 according to the present exemplary embodiment. FIG. 2 illustrates one element group 100, a circuit 200, and a terminal portion 300. FIG. 2 further illustrates a protection circuit 201, a first detection circuit 202, a second detection circuit 211, a delay circuit 212, an adding circuit 213, a transmission pulse input terminal 301, an output terminal 302 of the pulse echo signal, and an output terminal 303 of the photoacoustic wave signal. FIG. 2 further illustrates a transmission pulse 401, a signal 402 from the first element 102, a pulse echo signal 403, a reception signal 404 of the photoacoustic wave, a delayed application signal 405 of the photoacoustic wave reception, a delayed addition signal 406 of the pulse echo reception, and a photoacoustic wave signal 407.

The first element 102 and the plurality of second elements 103 are electrically separated from one another and are connected to the circuit 200. The circuit 200 is provided with the terminal portion 300 where signals are exchanged with the external apparatus, and the respective terminals 301, 302, and 303 in the terminal portion 300 are electrically separated from one another and are electrically connected to the cable 504. A cable obtained by binding together a plurality of fine-wire coaxial cables is used as the cable 504, and a connection to the external apparatus can be established via the connector 505 connected to the end of the cable 504. Hereinafter, an operation of the internal part of the probe 500 according to the present exemplary embodiment will be described by using FIG. 2.

First, the operation by using the pulse echo technique will be described. The first element 102 is electrically connected to the transmission pulse input terminal 301. The transmission pulse 401 for driving (high voltage transmission signal having several tens of volts to several hundreds of volts) is input to the transmission pulse input terminal 301 from the apparatus connected to the external part and can be directly applied to the first element 102. In addition, the protection circuit 201 is connected to the first element 102, and the high voltage transmission pulse applied to the input terminal 301 is not applied to the part beyond the protection circuit 201. For this reason, a situation is avoided where the first detection circuit 202 connected to the part beyond the protection circuit 201 is damaged by the high voltage transmission pulse.

The protection circuit 201 allows passage of a minute signal output from the first element 102, and the signal 402 output from the first element 102 when the first element 102 receives the ultrasonic wave or the photoacoustic wave is not blocked by the protection circuit 201 and is transmitted to the first detection circuit 202. The first detection circuit 202 amplifies the signal 402 from the first element 102 which has passed through the protection circuit 201 and outputs the voltage to the output terminal 302 of the pulse echo signal while the pulse echo signal 403 is set as the temporal waveform. At this time, the input of the transmission pulse 401 is detected by any device, and the output of the pulse echo signal is extracted on the basis of this detection result.

Herein, contour lines of the sound wave having at least a certain intensity when the ultrasonic wave is transmitted from the first element 102 are represented by curved lines U1 in FIG. 2. A width of the curved lines U1 becomes markedly wide when the acoustic lens 101 does not exist, and the intensity of the sound wave is decreased as a whole. On the other hand, when the acoustic lens 101 exists, the width of the X direction is narrowed as illustrated in FIG. 2, and the intensity of the ultrasonic wave transmitted from the first element 102 is increased in the area in front of the acoustic lens 101, and the sound wave converges. In a case where the acoustic lens 101 is used, the intensity of the sound wave on outer sides of the curved lines U1 is abruptly decreased, and the ultrasonic wave hardly reaches when a distance in the X direction is increased. A distance from the transmission and reception surface 503 in the area where the width between the curved lines U1 in the elevation direction (the X direction) in FIG. 2 is the narrowest is referred to as a focal distance, and a position thereof is referred to as a focal position (focus position).

When a reflector of the ultrasonic wave exists in the area where the ultrasonic wave is transmitted, the transmitted ultrasonic wave is reflected by this. When a size of the reflector is sufficiently small, the size of the ultrasonic wave reflected by the reflector is changed depending on the distance from the sound axis in the elevation direction (the X direction) of the reflector. Specifically, when the reflector is on the inner side of the curved lines U1, the reflector is irradiated with the intense ultrasonic wave, and the large reflected wave is returned. On the other hand, when the reflector is on the outer side of the curved lines U1, the ultrasonic wave becomes markedly weak, and the reflected wave is hardly returned. Since the reflected ultrasonic wave passes through the acoustic lens 101 to be returned to the first element 102, similarly as in the case of the transmission, a reflection signal from the reflector existing on the inner side of the curved lines U1 is detected with a high sensitivity, and a reflection signal from the reflector existing on the outer side of the curved lines U1 is detected with a low sensitivity.

Due to the two above-described reasons, the detected signal is changed depending on the position of the reflector in the X direction since the acoustic lens 101 exists, and the detection can be performed by selecting only the signal of the reflector in the area exactly opposite to the acoustic lens 101. According to the pulse echo technique, since the passage through the acoustic lens 101 occurs two times in total for the transmission and the reception, although depending on a method for the signal processing, in general, the slice resolution becomes approximately half of the width of the curved lines U1.

On the other hand, according to the photoacoustic imaging technique, since a wide area of a subject is uniformly irradiated with light, the area where the acoustic wave is generated is not to be decreased. Since the generated photoacoustic wave passes through the acoustic lens 101 only once, the slice resolution becomes approximately equal to the width of the curved lines U1. For this reason, since the slice resolution using the photoacoustic imaging technique is expanded as compared with the slice resolution using the pulse echo technique, even in a case where the same subject is measured, information of a different thickness is acquired.

According to the present exemplary embodiment, in order that the slice resolution using the photoacoustic imaging technique is set to be close to the slice resolution using the pulse echo technique, the detection is performed by using the detection signal from the plurality of second elements 103 in addition to the detection signal from the first element 102. Hereinafter, descriptions will be specifically given.

The different second detection circuit 211 is connected to each of the second elements 103, and the n second detection circuits 211 are provided to one element group 100 in the elevation direction. The second detection circuit 211 amplifies the signal output when the second element 103 receives the photoacoustic wave, and the voltage is output while the reception signal 404 of the photoacoustic wave is set as the temporal waveform. The delay circuit 212 is connected to an output terminal of each of the second detection circuits 211. The delay circuit 212 delays the analog temporal waveform 404 from the second detection circuit 211 by an arbitrary time and outputs the temporal waveform as the delayed application signal 405 of the photoacoustic wave reception. Delay times in respective delay circuits 205 are set to be different from one another on the basis of a distance X1 from the central element in the elevation direction (the X direction) of the connected second elements 103 and a focal distance Z1. Specifically, the respective delay times are set to have a value as close as possible to the delay time D=((Z1²+X1²)^{0.5}−Z1)/V by using the acoustic velocity V.

At this time, the detection signal (the pulse echo signal 403) from the first detection circuit 202 is output as the delayed addition signal 406 by the delay circuit 212 connected to the first detection circuit 202 and is input to the adding circuit 213. Subsequently, the plurality of waveform output signals (the delayed application signals 405) from the respective delay circuits 212 connected to the second detection circuits 211 are added to one another for each element group in the elevation direction in the adding circuit 213. Accordingly, the temporal waveform signal is output to the output terminal 303 of the photoacoustic detection signal as the photoacoustic wave signal 407. At this time, the light irradiation, the start of the photoacoustic imaging, the non-input of the transmission pulse, and the like are detected by any device, and the output of the photoacoustic wave signal is extracted on the basis of the detection result.

It is characterized in that the delay times in the respective delay circuits 212 connected to the second detection circuits 211 are set such that the phase difference disappears when the signal has arrived from the same position as the focal position of the acoustic lens 101. For this reason, the delayed addition signal 406 of the waveform output from the first detection circuit 202 is added to the plurality of delayed application signals 405 of the waveform outputs from the respective delay circuits 212 connected to the second detection circuits 211, so that the following effect can be attained. That is, it is possible to attain the same effect (for example, a sound pressure distribution of curved lines U2 in FIG. 2) as the case of the arrangement of the acoustic lens having the same opening area as the width (width obtained by adding the regions P, S, and Q to one another) of the element group in the elevation direction. Since the slice resolution in the position at the focal distance corresponds to (the depth x the wavelength)/the opening area (the distance in the perpendicular direction from the probe surface corresponds to the depth), the slice resolution can be improved to W2 as compared with the slice resolution (W1) based on only the reception when the acoustic lens having the width S is used. This slice resolution is uniformly improved in the area exactly opposed to the acoustic lens 101, and the slice resolution using the photoacoustic imaging technique can be set to be close to the same value at the time of the signal detection using the pulse echo technique. For this reason, the slice resolution W2 is preferably set to be approximately twice as high as W1.

It is noted that the area of the region S (which corresponds to the area of the region where the acoustic lens 101 is arranged on the transmission and reception surface 503) is preferably substantially equal to a sum of the area of the region P and the area of the region Q (which correspond to the areas of the regions where the acoustic lens 101 is not arranged in the transmission and reception surface 503). Accordingly, since the slice resolution is determined on the basis of the width of the entire elements in the X direction (in general, which is referred to as an opening width), the slice resolution at the time of the transmission and reception can be substantially matched with the slice resolution at the time of the photoacoustic wave reception. A state in which the area of the region S is substantially equal to the sum of the area of the region P and the area of the region Q means that the area of the region S is 0.8 times or more but 1.2 times or less as high as the sum of the area of the region P and the area of the region Q. In this manner, the area of the region S, the area of the region P, and the area of the region Q preferably satisfy a relationship in which the slice resolution at the time of the transmission and reception is substantially matched with the slice resolution at the time of the photoacoustic wave reception.

Herein, consideration will be given to another method (comparison example) of improving the slice resolution. As the other method, a method of simply expanding the opening of the acoustic lens is conceivable. In general, the intensity of the acoustic lens used for the search unit attenuates when the ultrasonic wave or the photoacoustic wave passes therethrough. For this reason, when the opening of the acoustic lens is expanded at the same curvature, since the thickness of the acoustic lens is increased as a whole, the slice resolution is improved, but the signal intensity itself is decreased. On the other hand, when the configuration of the above-described search unit is used, the thickness of the acoustic lens does not need to be increased. The attenuation in the acoustic lens is not increased, and the intensity degradation of the photoacoustic wave that reaches the element hardly occurs, so that the slice resolution using the photoacoustic imaging technique can be improved.

As the transducer of the search unit according to the present exemplary embodiment, a device configured to output a voltage signal corresponding to a received ultrasonic wave when an ultrasonic wave of a piezoelectric transducer (PZT), polymer membrane (PVDF), or the like is received can be used. As the first detection circuit 202 and the second detection circuit 211 for the fine voltage signal output from the search unit, a voltage amplification type preamplifier (voltage amplification circuit) can be used. An analog LC delay circuit using an inductor or a capacitance, a CCD delay circuit using charge-coupled devices in multiple stages, a digital delay circuit using an analog-to-digital converter, a digital signal processing circuit, a digital-to-analog converter, and the like can be used as the delay circuit 212 according to the present exemplary embodiment.

According to the present exemplary embodiment, the attenuation in the acoustic lens is not increased, and the intensity degradation of the photoacoustic wave that reaches the element hardly occurs, so that it is possible to improve the slice resolution using the photoacoustic imaging technique. For this reason, it is possible to provide the search unit and the probe in which the degradation in the characteristic of the detection for the ultrasonic wave and the photoacoustic wave by using the pulse echo technique and the photoacoustic imaging technique is small, and also the slice resolutions of those are close to each other. The search unit according to the present exemplary embodiment can also be similarly used as a general search unit using the pulse echo technique. Therefore, a general-purpose subject information acquiring apparatus can be used without the necessity of a significant change since the detection signal processed for each element group 100 by using the photoacoustic imaging technique is output. In addition, the signal is acquired by employing the same element using the pulse echo technique and the photoacoustic imaging technique. For this reason, when the signals of the same subject are acquired by using the pulse echo technique and the photoacoustic imaging technique, the occurrence of the relative positional deviation of the respective signal information with respect to the subject can be suppressed, so that it is possible to collect a large amount of information.

### Second Exemplary Embodiment

The present exemplary embodiment is different from the first exemplary embodiment in that one signal output multipurpose terminal 304 plays the roles as the output terminal 302 used for the detection of the ultrasonic wave and the output terminal 303 used for the detection of the photoacoustic wave. The other configurations are the same as those of the first exemplary embodiment.

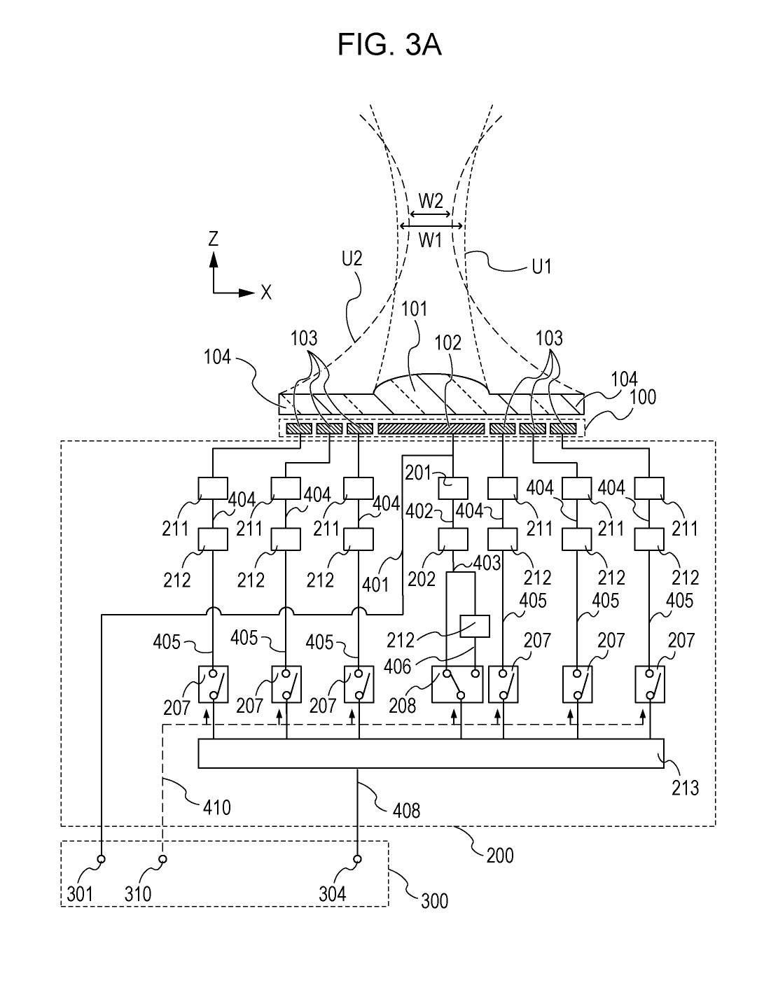

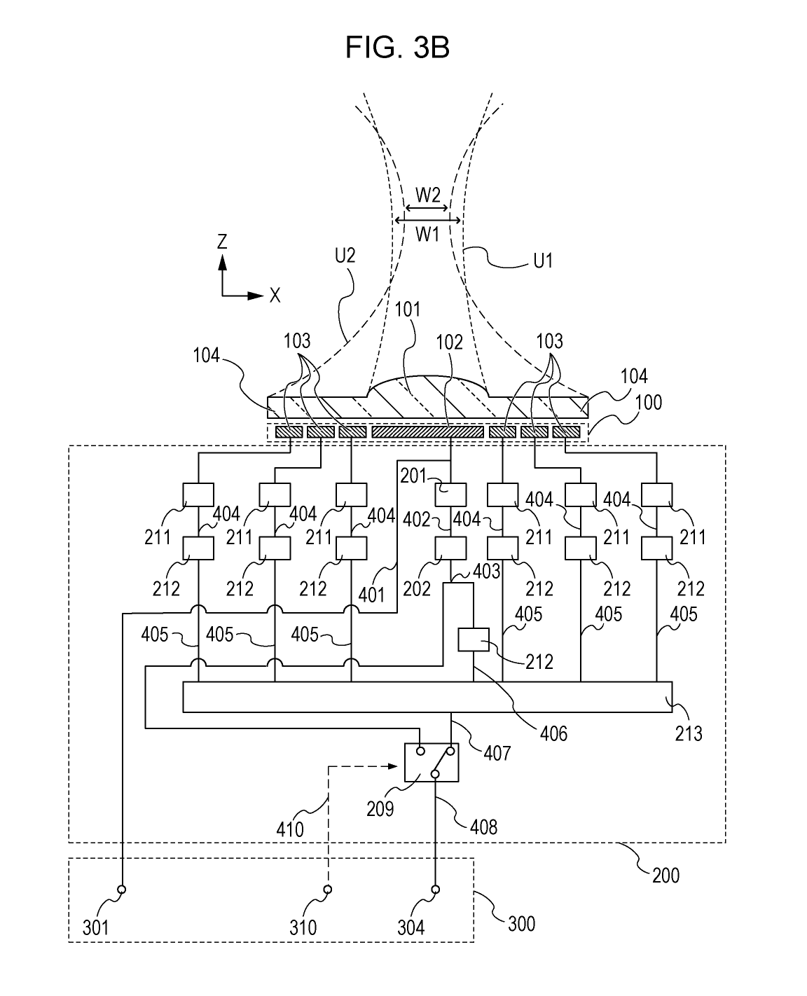

FIG. 3A and FIG. 3B are schematic views for describing an element, a circuit, and a terminal in the probe 500 according to the present exemplary embodiment. FIG. 3A illustrates a first switch 208, a second switch 207, the signal output multipurpose terminal 304, a switch switching signal terminal 310, and a switch switching signal 410 according to a first mode example of the present exemplary embodiment. According to the first mode example, the signal output multipurpose terminal 304 is provided instead of the output terminal 302 of the pulse echo signal and the output terminal 303 of the photoacoustic wave signal according to the first exemplary embodiment.

In FIG. 3A according to the first mode example, the first switch 208 is arranged between the delay circuit 212 connected to the first detection circuit 202 and the adding circuit 213. The second switch 207 is connected between the delay circuit 212 connected to the second detection circuit 211 and the adding circuit 213. Switching of the first switch 208 and the second switch 207 is performed by the switch switching signal 410 from the switch switching signal terminal 310. When the pulse echo operation is performed, the first switch 208 is switched by the switch switching signal 410 such that the first detection circuit 202 is directly connected to the adding circuit 213. On the other hand, the lines are put into a non-connection state by the second switch 207, and no signal is input to the adding circuit 213 from the delay circuit 212 connected to the second detection circuit 211. For this reason, only the pulse echo signal 403 from the first detection circuit 202 is input to the adding circuit 213, and the pulse echo signal 403 is output to the signal output multipurpose terminal 304 as it is as a signal output 408 without being added with the other signals. The signal output multipurpose terminal 304 is provided for each of the element groups.

On the other hand, when the photoacoustic operation is performed, the switching of the first switch 208 is performed by the switch switching signal 410 such that the delay circuit 212 connected to the first detection circuit 202 is directly connected to the adding circuit 213. The lines are put into a connection state by the second switch 207, and the signal is input to the adding circuit 213 from the delay circuit 212 connected to the second detection circuit 211. For this reason, the delayed addition signal 406 of the detection signal by the first detection circuit 202 is added to the delayed application signal 405 of the photoacoustic wave reception in the adding circuit 213, and the same signal as the photoacoustic wave signal 407 of FIG. 2 is output to the signal output multipurpose terminal 304 as the signal output 408.

The second switches 207 and the first switch 208 according to the present mode example are provided by n corresponding to the number of the second elements and 1 corresponding to the number of the first element for the element group 100 in the elevation direction, and the {(n+1)×m} pieces of the second switches 207 and the first switches 208 are provided in the probe 500 including the m sets of the element groups 100 in the elevation direction. Since it is sufficient when the second switch 207 and the first switch 208 have a function of turning ON or OFF of the passage of the detection signal or a function of a multiplexer configured to switch the lines of 2:1, the configuration can be easily made by using a low voltage analog switch.

With the above-described configuration, it is possible to generate the switch switching signal 410 while the signals are respectively generated in accordance with the pulse echo operation and the photoacoustic operation in the apparatus connected to the external part. In addition, the same switch switching signal can be used for all sets of the element groups 100 in the elevation direction in the ultrasonic wave the probe 500. For this reason, since the number of signals input to the probe 500 is increased by only one, the above-described function can be realized while the configuration of the external apparatus is hardly changed.

Since the probe 500 according to the present mode example can play the parts as the output terminal 302 of the pulse echo signal and the output terminal 303 of the photoacoustic wave signal, the number of lines of the signal outputs in the cable 504 can be decreased from (2×m) to (m+1). For this reason, the diameter of the cable 504 can be decreased, and the flexibility of the cable is increased. Accordingly, it is possible to provide the probe that can be used for both the transmission and reception using the pulse echo technique and the reception using the photoacoustic imaging technique and has a satisfactory operability. Since the number of lines is also decreased, the size of the connector 505 can be reduced, and the probe 500 can be downsized. In addition, the number of lines that need to be provided to the connected apparatus is decreased, the configuration of the apparatus at the connection destination can be simplified. It is noted that the second switch 207 and the first switch 208 are arranged between the delay circuit 212 and the adding circuit 213 according to the present mode example, but the configuration is not limited to this. A configuration in which the second switch 207 and the first switch 208 are arranged between the detection circuit and the delay circuit or arranged in a stage preceding the detection circuit can also be adopted.

Another mode example of the present exemplary embodiment will be described by using FIG. 3B. According to this another mode example, a switch 209 is provided so that this switch selects the first detection circuit 202 or the adding circuit 213 as the circuit that inputs the signal to the signal output multipurpose terminal 304. It is sufficient when only m pieces of the switches 209 may be provided corresponding to the number of the element groups 100 in the Y direction corresponding to the elevation direction.

The switch 209 switches, on the basis of the switch switching signal input via the switch switching signal terminal 310, the pulse echo signal 403 from the first detection circuit 202 or the photoacoustic wave signal 407 from the adding circuit 213 to be connected to the signal output multipurpose terminal 304. The output of the first detection circuit 202 (the pulse echo signal 403) is connected to the signal output multipurpose terminal 304 when the signal is acquired by using the pulse echo technique on the basis of the switch switching signal from the switch switching signal terminal 310. On the other hand, when the signal is acquired by using the photoacoustic imaging technique, the output of the adding circuit 213 (the photoacoustic wave signal 407) is connected to the signal output multipurpose terminal 304.

When the above-described another mode example is used, since the number of switches can be significantly decreased from {(n+1)×m} to m as compared with the configuration of FIG. 3A, the configuration of the probe 500 can be simplified and downsized.

### Third Exemplary Embodiment

According to the present exemplary embodiment, it is characterized in that one signal input/output terminal is provided to the element group 100 in the elevation direction. The other configurations are the same as those of the second exemplary embodiment described by using FIG. 3A and FIG. 3B.

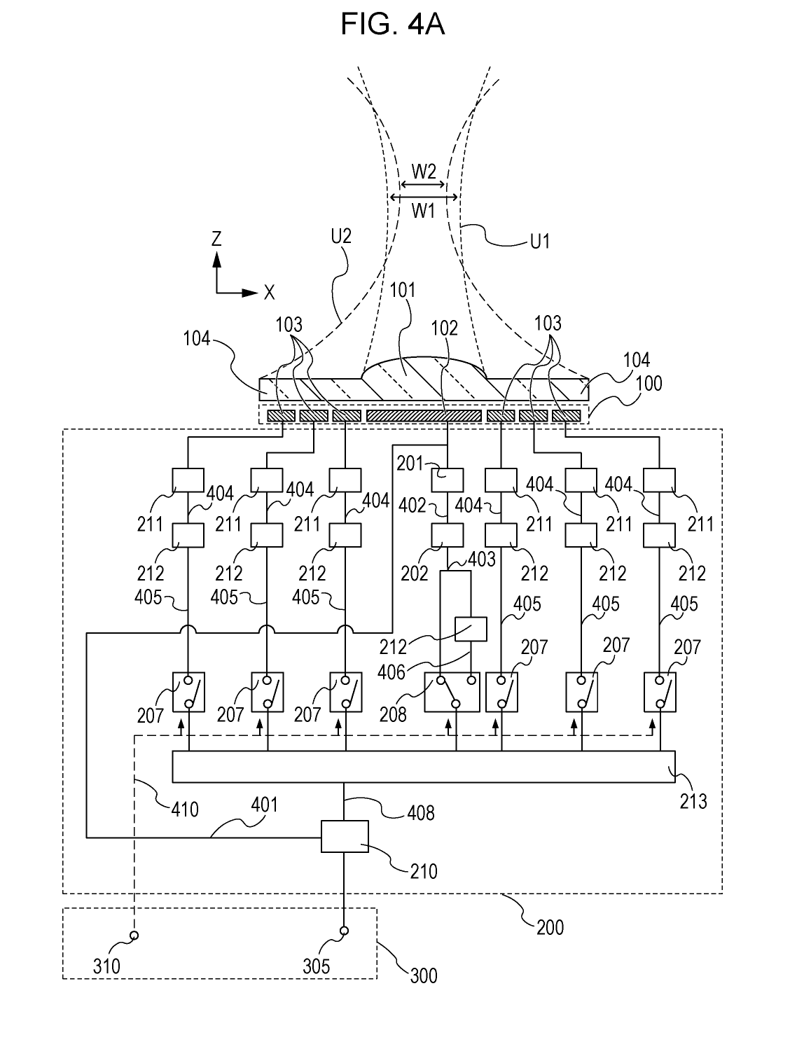

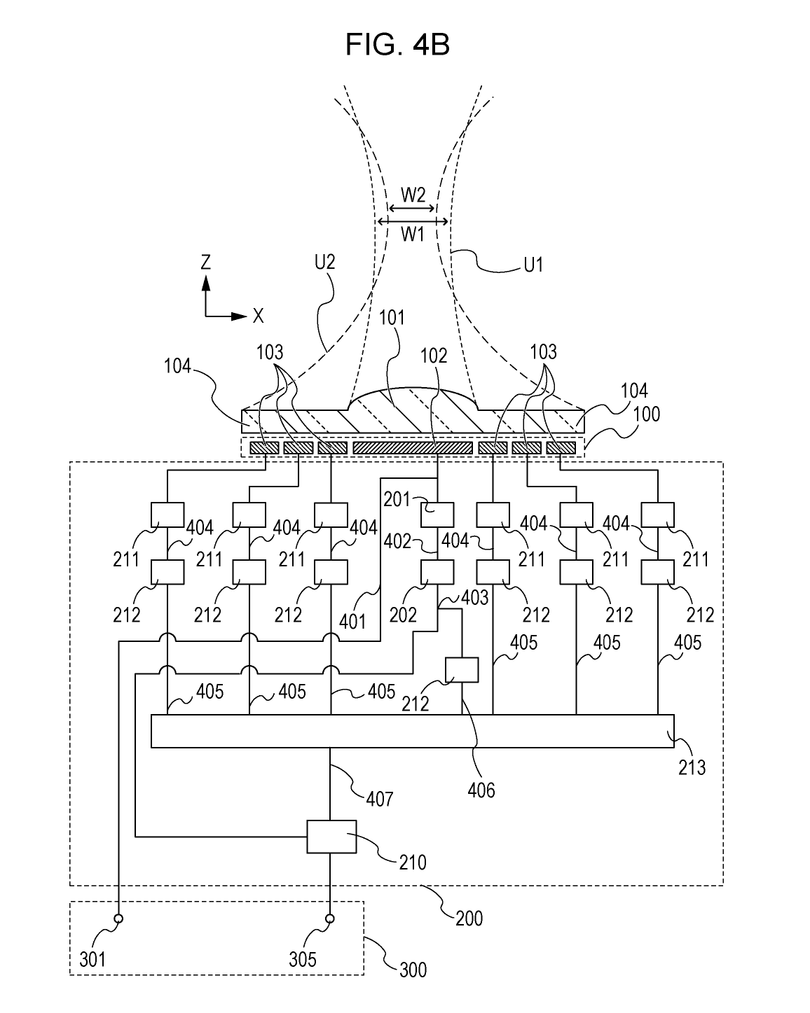

FIG. 4A and FIG. 4B are schematic views for describing the element, the circuit, and the terminal in the probe 500 according to the present exemplary embodiment. FIG. 4A illustrates an input/output multipurpose terminal 305 and a transmission/reception signal selection unit 210 according to a first mode example. According to the present mode example, the input/output multipurpose terminal 305 plays the roles as three terminals for the input of the transmission pulse 401 to the probe 500, the output of the pulse echo signal 403 from the probe 500, and the output of the photoacoustic wave signal 407 from the probe 500. The transmission/reception signal selection unit 210 is connected to the input/output multipurpose terminal 305, and the transmission/reception signal selection unit 210 is connected to the line that is connected to the first element 102 and the protection circuit 201 and the line that is connected to the adding circuit 213.

When the input/output multipurpose terminal 305 is applied with the transmission pulse 401, the transmission/reception signal selection unit 210 transmits the transmission pulse 401 to the line connected to the first element 102 and the protection circuit 201 on the basis of the detection result. At this time, the transmission/reception signal selection unit 210 does not transmit the transmission pulse 401 to the line connected to the adding circuit 213. For this reason, it is possible to avoid the damage of the adding circuit 213 caused by the high voltage transmission pulse 401. In a case where the transmission/reception signal selection unit 210 is not provided with a shield unit that protects the adding circuit 213 from the transmission pulse 401, the output stage of the adding circuit 213 needs to have a configuration using a plurality of large elements resistant to a high voltage, the size of the circuit 200 as a whole is increased. According to the present mode example, the above-described situation is avoided.

On the other hand, when the input/output multipurpose terminal 305 is not applied with the transmission pulse 401, the transmission/reception signal selection unit 210 transmits the signal from the line connected to the adding circuit 213 (the detection signal output 408) to the input/output multipurpose terminal 305. At this time, the transmission/reception signal selection unit 210 does not transmit the detection signal output 408 to the line connected to the first element 102 and the protection circuit 201. Accordingly, it is possible to avoid the situation where the output signal is applied to the first detection circuit 202 again, and the signal is amplified to infinity to cause the oscillation or the distortion of the detection signal. The transmission/reception signal selection unit 210 can be easily realized by using an analog switch or the like which is configured to perform line switching in accordance with the presence or absence of the high voltage transmission pulse 401 and is resistant to the high voltage without increasing the circuit area so much.

Hereinafter, the signal at the time of the pulse echo operation and the signal at the time of the photoacoustic operation will be described. At the time of the pulse echo operation, first, the transmission pulse 401 is applied to the input/output multipurpose terminal 305 from the external apparatus via the connector 505 and the cable 504. Since the transmission/reception signal selection unit 210 transmits the transmission pulse 401 to the first element 102, it is possible to transmit the ultrasonic wave from the first element 102. At this time, since the switch switching signal 410 turns to the signal representing the pulse echo operation, the detection signal from the first element 102 (the pulse echo signal 403) is transmitted to the input/output multipurpose terminal 305 from the adding circuit 213.

On the other hand, at the time of the photoacoustic operation, the switch switching signal 410 turns to the signal representing the photoacoustic operation, and the signals (the photoacoustic wave signals) acquired by respectively appropriately delaying the detection signals from the first element 102 and the second element 103 are transmitted to the input/output multipurpose terminal 305 from the adding circuit 213.

According to the present exemplary embodiment, since the transmission/reception signal selection unit 210 is provided, this can be used as both the terminal to which the transmission pulse 401 is input and the terminal from which the pulse echo signal 403 and the photoacoustic wave signal 407 are output. For this reason, the number of lines used for the transmission of the transmission/reception signals in the cable 504 can be set as m, and the cable can be made more flexible. Therefore, it is possible to provide the probe with a more satisfactory operability.

According to the present exemplary embodiment, since it is sufficient when m pieces of the transmission/reception signal selection units 210 are added for each element group 100 in the elevation direction, the size of the entire circuit is hardly changed. In addition, since the number of lines is further decreased, the size of the connector 505 is further reduced, so that the probe 500 can be further downsized. In addition, the number of terminals that need to be provided to the connected apparatus is also decreased, it is also possible to further simplify the configuration of the apparatus at the connection destination. Since the apparatus connected to the probe generally has the configuration of using the multipurpose transmission/reception terminal, when the probe 500 according to the present exemplary embodiment is used, the configuration of the transmission/reception terminal on the connected apparatus side does not need to be changed from the general apparatus. For this reason, the apparatus can be used by only adding the generation unit for the switch switching signal 410 to the general-use apparatus.

It is noted that the descriptions have been given of the mode example described in FIG. 4A on the basis of the circuit configuration of FIG. 3A, but the configuration is not limited to this. The same can also similarly be applied to the circuit configuration of FIG. 3B. That is, in FIG. 2, the two output terminals 302 and 303 are set as the input/output multipurpose terminal 305, and the transmission/reception signal selection unit 210 is added, the configuration of FIG. 4B can also similarly be adopted.

### Fourth Exemplary Embodiment

It is characterized in the present exemplary embodiment that a unit configured to detect the transmission pulse applied when the pulse echo technique is employed is provided. The other configurations are the same as those of the third exemplary embodiment.

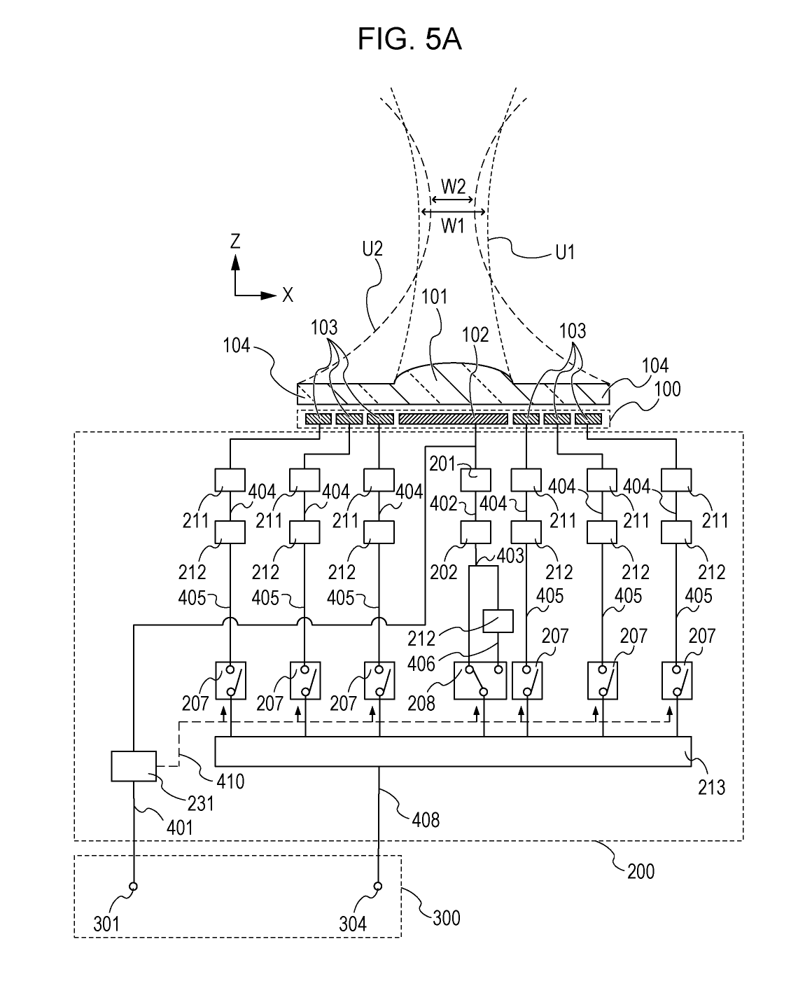

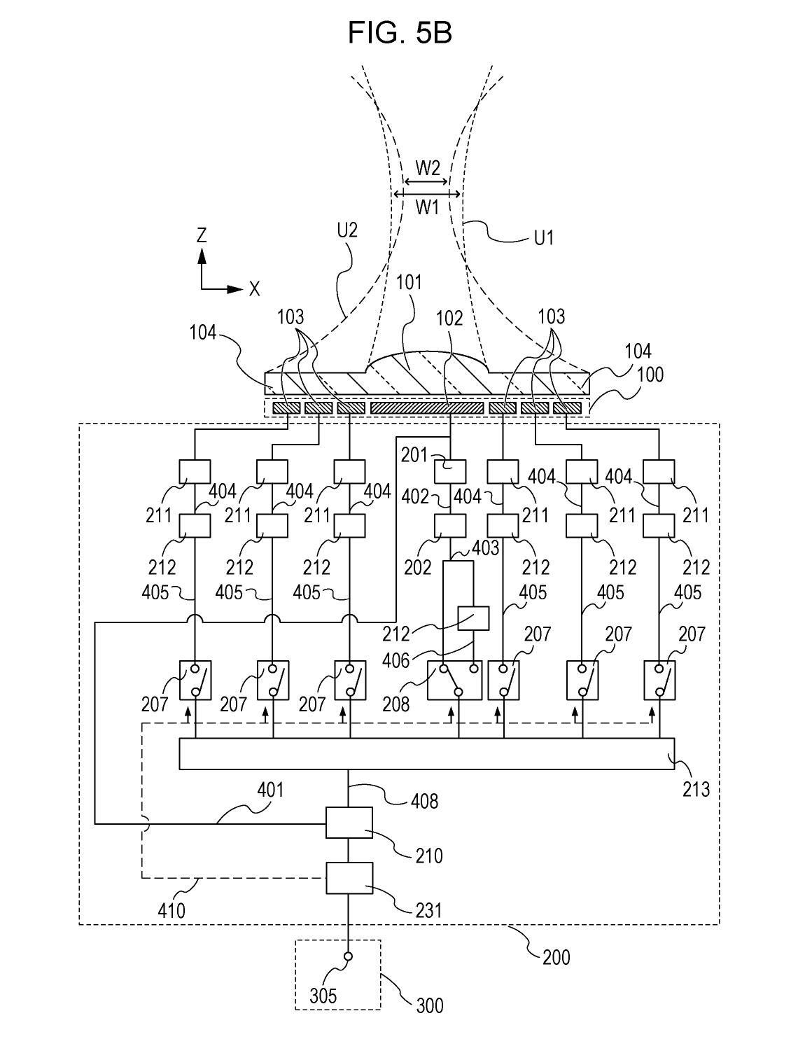

FIG. 5A and FIG. 5B are schematic views for describing the element, the circuit, and the terminal in the probe 500 according to the present exemplary embodiment. FIG. 5A illustrates a transmission pulse detection unit 231. FIG. 5A illustrates a mode in which the probe described in FIG. 3A is applied to the present exemplary embodiment. The transmission pulse detection unit 231 is arranged on a route on the line through which the transmission pulse 401 passes. When the applied voltage exceeds a predetermined voltage VP, the transmission pulse detection unit 231 outputs the signal representing the pulse echo operation as the switch switching signal 410. Herein, the predetermined voltage VP is generally set as approximately 1 V to 5 V. Since the transmission pulse detection unit 231 is provided, it is possible to detect the timing at which the probe 500 is applied with the transmission pulse 401. That is, without the switch switching signal from the connected apparatus 410, it is possible to detect that the signal acquisition for the image formation is performed by the probe 500 on the basis of the pulse echo technique.

The transmission pulse detection unit 231 continues outputting the signal representing the pulse echo operation for a certain period since the transmission pulse 401 is detected. This period is set to be longer than a time when the transmitted ultrasonic wave is reflected by the reflector to be returned. Specifically, this period is set on the basis of a depth on the subject side detected by way of the pulse echo technique and the sound speed of the ultrasonic wave. The switch switching signal 410 output from the transmission pulse detection unit 231 is input to the second switch 207 and the first switch 208, and the pulse echo operation described in FIG. 3A is performed.

After the elapse of the certain period since the transmission pulse 401 is detected, the transmission pulse detection unit 231 continues outputting the signal representing the photoacoustic operation as the switch switching signal 410 unless the transmission pulse 401 is detected. Therefore, at this time, the probe 500 according to the present exemplary embodiment performs the photoacoustic operation described in FIG. 3A.

When the above-described mode example of the present exemplary embodiment is used, it is possible to detect that the signal acquisition for the image formation is performed by the probe 500 on the basis of the pulse echo technique without the switch switching signal from the connected apparatus 410. For this reason, the connected apparatus does not need to generate the switch switching signal 410, and it is not necessary to add a special output function for the control signal to the apparatus.

Next, another mode of the present exemplary embodiment will be described. FIG. 5B illustrates a mode in which the probe described in FIG. 4A is applied to the present exemplary embodiment. It is characterized in FIG. 5B that the transmission pulse detection unit 231 is provided between the input/output multipurpose terminal 305 and the transmission/reception signal selection unit 210.

When the transmission pulse 401 is not input from the input/output multipurpose terminal 305, the transmission pulse detection unit 231 transmits the detection signal output 408 that has been output from the adding circuit 213 and passed through the switch (the transmission/reception signal selection unit 210) to the input/output multipurpose terminal 305 as it is. On the other hand, when the transmission pulse 401 is input from the input/output multipurpose terminal 305, the signal representing the pulse echo operation is output as the switch switching signal 410. The other operations are the same as those described by using FIG. 5A.

Since the probe according to the present exemplary embodiment is provided with the transmission pulse detection unit 231, the switch switching signal from the connected apparatus is not necessary. Furthermore, since the transmission/reception signal selection unit 210 is provided, a terminal can be used as both the terminal to which transmission pulse 401 is input and the terminal from which the photoacoustic wave signal is output. For this reason, since it is sufficient when the number of signal lines that connect the probe to the external apparatus is only m corresponding to the number of sets of the element groups 100 in the elevation direction, the flexibility of the probe cable can be markedly increased. In addition, since only one transmission/reception line is provided for each of the elements similarly as in the general probe, the general-use ultrasonic wave apparatus can be used as it is.

It is noted that the descriptions have been given on the basis of the circuit configuration of FIG. 3A according to the present exemplary embodiment, but the configuration is not limited to this. The configuration can also be used for the circuit configuration of FIG. 3B. That is, it is also possible to adopt a configuration in which the transmission pulse detection unit 231 is provided to the line route through which the transmission pulse 401 of FIG. 3B passes, and the transmission pulse detection unit 231 controls the switch 209 on the basis of the switch switching signal 410.

### Fifth Exemplary Embodiment

According to the present exemplary embodiment, a type of the search unit is different. The other configurations are the same as those of any of the first to fourth exemplary embodiments.

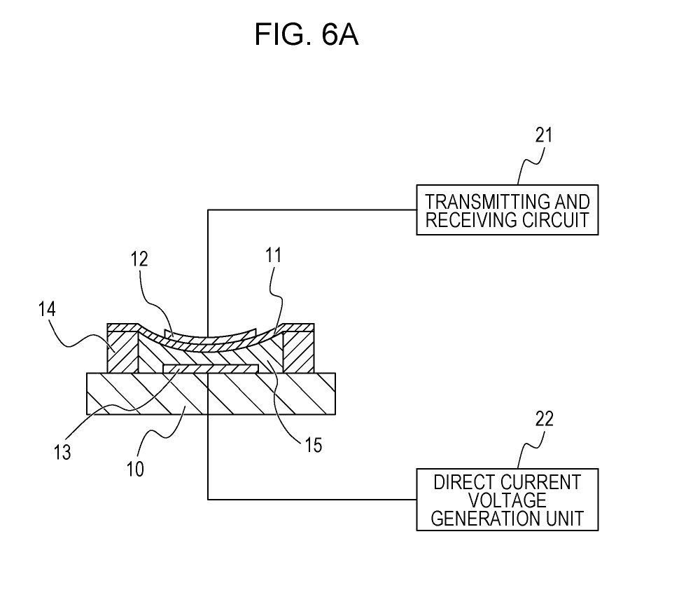

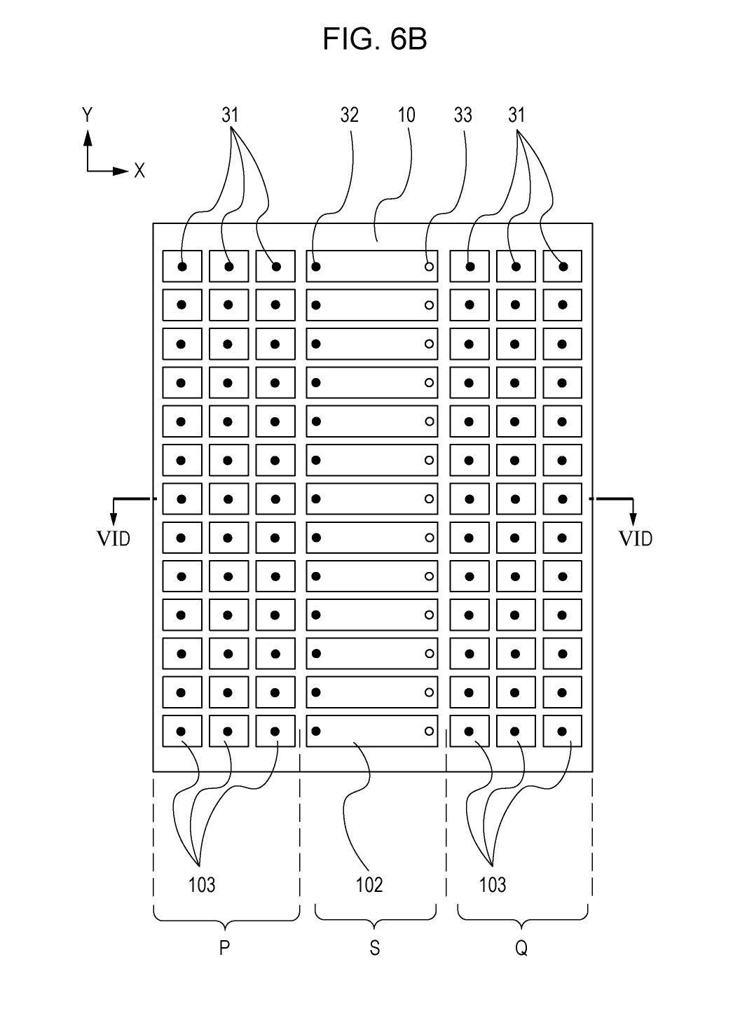

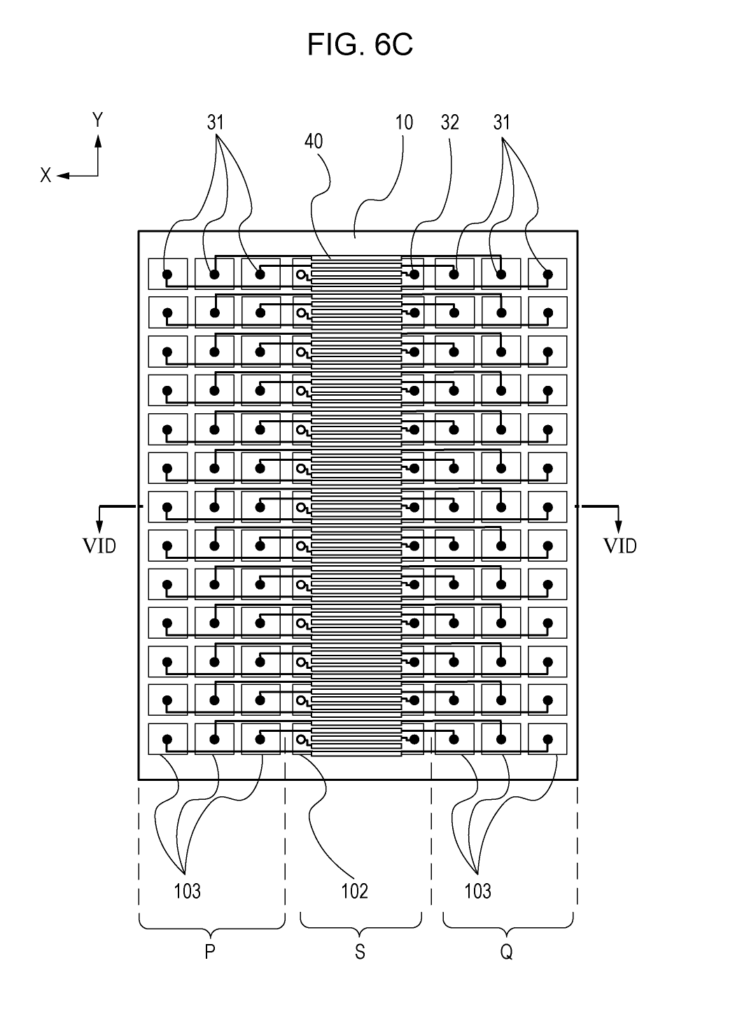

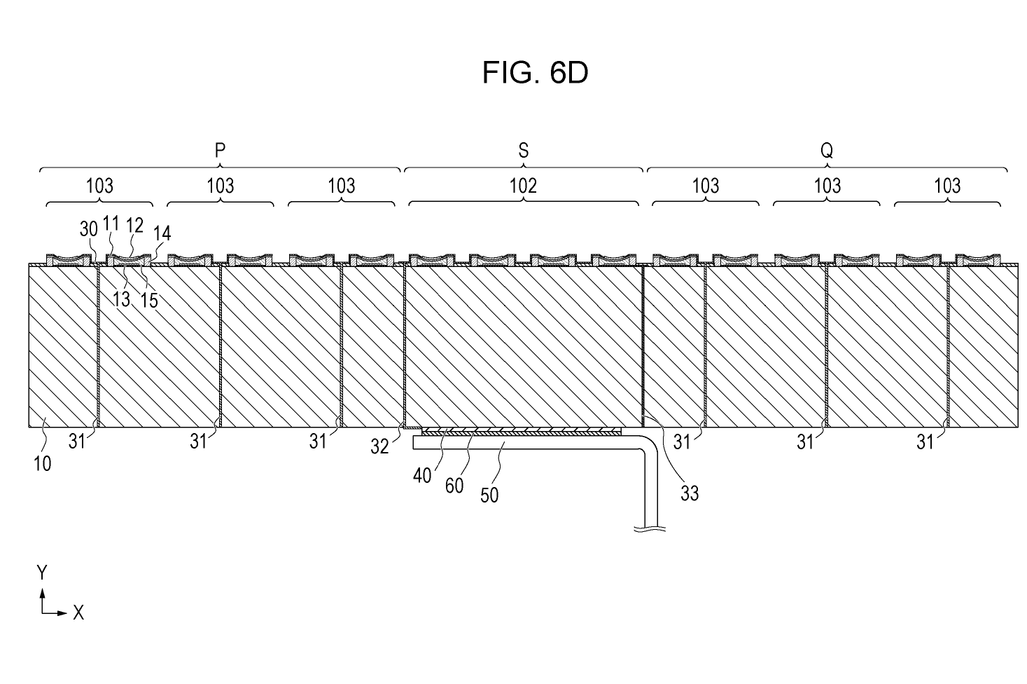

According to the present exemplary embodiment, it is characterized in that a CMUT is used as the search unit. FIG. 6A, FIG. 6B, FIG. 6C, and FIG. 6D are schematic views for describing the search unit (CMUT) according to the present exemplary embodiment. FIG. 6A illustrates a substrate 10, a vibration film 11, a first electrode 12, a second electrode 13, a supporting portion 14, a cavity 15, a direct current voltage generation unit 22, and a transmitting and receiving circuit 21. FIG. 6B, FIG. 6C, and FIG. 6D illustrate a wiring 30, a first penetrating through hole 31, a second penetrating through hole 32, a third penetrating through hole 33, a connecting electrode 40, a flexible printed wiring 50, and an electric connecting portion 60.

A capacitive micromachined ultrasonic transducer (CMUT) corresponding to an electrostatic capacitance type transducer has been proposed for the purpose of the transmission and reception of the ultrasonic wave. The CMUT is manufactured by using a micro electro mechanical systems (MEMS) process realized by the application of a semiconductor process. The CMUT has such characteristics that a band of a frequency at the time of the transmission and reception is wide, and a reproducibility of the signal is satisfactory as compared with a piezoelectric transducer. In the CMUT (transmitting and receiving element) illustrated in FIG. 6A, a group of the first electrode 12 and the second electrode 13 facing each other with the vibration film 11 and the cavity 15 therebetween is referred to as a cell. The vibration film 11 is supported by the supporting portion 14 formed on the substrate (chip) 10 such that the vibration film 11 can be vibrated. The direct current voltage generation unit 22 is connected to the second electrode 13 to be applied with a predetermined direct current voltage Va. The first electrode 12 is connected to the transmitting and receiving circuit 21.

On the other hand, the first electrodes 12 are electrically connected to one another for every plural cells provided to an element unit (element) of the CMUT. The first electrodes 12 are connected to the transmitting and receiving circuit 21 respectively for each element. A potential of the first electrode 12 is set as a fixed potential in the proximity of a GND potential by the transmitting and receiving circuit 21. Accordingly, a potential difference of Vbias=Va−0 V is generated between the first electrode 12 and the second electrode 13. By adjusting a value of Va, a value of Vbias is matched with a desired potential difference (approximately several tens of V to several hundreds of V) which is determined by a mechanical characteristic of the cell of the CMUT.

While the first electrode 12 is applied with an alternating current driving voltage by the transmitting and receiving circuit 21, an alternating current electrostatic attraction force is generated between the first and second electrodes, and the ultrasonic wave can be transmitted by vibrating the vibration film 11 at a certain frequency. In addition, since the vibration film 11 is vibrated by receiving the ultrasonic wave, a minute current is generated in the first electrode 12 by an electrostatic induction, and a current value thereof is measured by the transmitting and receiving circuit 21 to take out the reception signal.

FIG. 6B is a schematic view of the search unit (CMUT) 502 according to the present exemplary embodiment as seen in a direction from the transmission and reception surface 503 side to the inner side of the probe 500. As may be understood by also referring to FIG. 6D, the first element 102 includes one each of the second penetrating through hole 32 and the third penetrating through hole 33. The second penetrating through hole 32 and the third penetrating through hole 33 are arranged at both ends in the elevation direction (the X direction) of the first element 102. Each of the second elements 103 has the one first penetrating through hole 31. The first penetrating through hole 31 is arranged in the vicinity of the center of the second element 103. According to the present exemplary embodiment, since the second penetrating through hole 32 and the third penetrating through hole 33 of the first element 102 are arranged at both ends of the first element 102, it is possible to secure a region where the connecting electrode is arranged.

FIG. 6C is a schematic view of the search unit (CMUT) 502 according to the present exemplary embodiment as seen from the back side of FIG. 6B. The connecting electrode 40 is arranged at the center of the substrate back surface in the region of the first element 102. The two connecting electrodes 40 are connected to the first element 102, and the (n/2) pieces of connecting electrodes 40 are connected to the second element 103 per set of the element groups 100 in the elevation direction.

A group of the first penetrating through holes 31 of the second elements 103 located at the left and right sides in the order corresponding to the closer positions to the first element 102 are connected to the same connecting electrode 40. When the intensity distributions of the sound pressures become symmetrical to each other with respect to the front side of the probe are realized, the same phase difference may be applied to the detection signals in the group of the second elements 103 at the left and right sides in the order corresponding to the closer positions to the first element 102. The CMUT is the current outputting element, and when the electrodes are connected to each other, the current is added before being input to the detection circuit, so that the above-described configuration can be realized. Accordingly, the numbers of the second detection circuits 211 and the delay circuits 212 connected to the second detection circuits 211 (for example, see FIG. 2) can be set as half the number of n (the number of second elements of the element group 100), and the circuit area can be reduced.

FIG. 6D is a cross-sectional view of the search unit (CMUT) 502 according to the present exemplary embodiment as seen from the VID-VID cross section of FIG. 6B. The second penetrating through hole 32 is connected to the first electrode 12 of the first element 102. The second electrode 13 of the first element 102 is connected to the third penetrating through hole 33 and connected to the direct current voltage generation unit 22. The second electrodes 13 provided to the plurality of second elements 103 are electrically connected to one another (not illustrated in FIG. 6D). The first electrode 12 is connected to the first penetrating through hole 31 via the wiring 30 and connected to the transmitting and receiving circuit 21.

According to the present exemplary embodiment, the flexible printed wiring 50 is used for the connection between the search unit 502 and the circuit 200. The flexible printed wiring 50 has an electrode pattern corresponding to the connecting electrode 40 on the back surface of the search unit 502. The flexible printed wiring 50 is connected to the connecting electrode 40 by the electric connecting portion 60. Any portion may be used as the electric connecting portion 60 as long as the portion can electrically separate the adjacent connecting electrode 40 from each other and can electrically connect the flexible printed wiring 50 to the connecting electrode 40. For example, the portion can be easily realized by using anisotropically-conductive resin (ACF, ACP), soldering, or bump junction.

When the present exemplary embodiment is employed, since the configuration used for the connection with the circuit 200 can be arranged at the central part of the search unit 502, it is not necessary to provide a scape in a surrounding part of the search unit 502 for the connection with the circuit 200. In addition, the signal in the wide frequency range can be transmitted and received since the CMUT is used, and it is possible to acquire much information from the subject. Accordingly, it is possible to provide the probe having the small size in the surrounding of the transmission and reception surface 503 with the satisfactory operability in which the information acquiring amount is high.

In the above, for the explanation, the direct current voltage generation unit 22 is connected to the second electrode 13, and the first electrode 12 is connected to the transmitting and receiving circuit 21, but a configuration may also be similarly used in which the direct current voltage generation unit 22 is connected to the first electrode 12, and the second electrode 13 is connected to the transmitting and receiving circuit 21. At this time, the second electrode 13 is preferably connected to the third penetrating through hole 33, and the first electrode 12 is preferably connected to the first penetrating through hole 31.

It is noted that the detection circuit, the delay circuit, the adding circuit, and the switch described according to the first to fifth exemplary embodiments can be realized by an integrated circuit. In a case where the circuits of the respective exemplary embodiments are constituted by the integrated circuit, the circuit area can be significantly reduced, and it is possible to provide the small-sized highly functional probe.

### Sixth Exemplary Embodiment

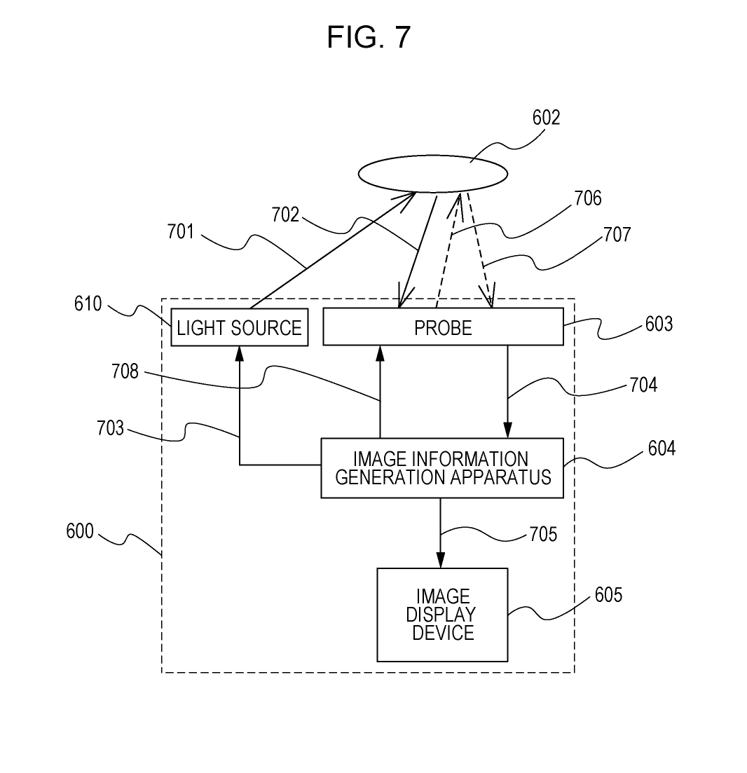

According to the present exemplary embodiment, the probe according to any one of the first to fifth exemplary embodiments is used as the subject information acquiring apparatus. FIG. 7 is a schematic view of the subject information acquiring apparatus according to the present exemplary embodiment. FIG. 7 illustrates an information acquiring apparatus 600, a light source 610, a subject 602, a probe 603, an image information generation apparatus 604 corresponding to an acquiring unit configured to acquire information of the subject, and an image display device 605. FIG. 7 also illustrates a light emission instruction signal 703, light 701, an ultrasonic wave 702 based on a photoacoustic signal, a photoacoustic signal (ultrasonic wave reception signal) 704, and reproduction image information 705.

The operation of the information acquiring apparatus according to the present exemplary embodiment will be described. First, a situation at the time of the pulse echo (ultrasonic wave transmission and reception) operation will be described. The ultrasonic wave 706 is output (transmitted) from the probe 603 toward the subject 602. The ultrasonic wave is reflected on the surface of the subject 602 due to a difference in specific acoustic impedances at an interface thereof. A reflected ultrasonic wave 707 is received by the probe 603, and information such as a size and a shape of the reception signal and time is transmitted to the image information generation apparatus 604 as the ultrasonic wave reception signal 704. On the other hand, the information such as a size and a shape of the transmission ultrasonic wave and time is stored in the image information generation apparatus 604 as information of an ultrasonic wave transmission signal 708. The image information generation apparatus 604 generates an image signal of the subject 602 on the basis of the information of the ultrasonic wave reception signal 704 and the ultrasonic wave transmission signal 708 to be output as the reproduction image information 705.

At the time of the photoacoustic operation, first, the light 701 (pulse light) is generated from the light source 610 on the basis of the light emission instruction signal 703, and the subject 602 is irradiated with the light 701. The photoacoustic wave (the ultrasonic wave) 702 is generated in the subject 602 through the irradiation of the light 701, and the ultrasonic wave 702 is received by the probe 603. The information such as the size and the shape of the reception signal and the time is transmitted to the image information generation apparatus 604 as the ultrasonic wave reception signal 704. On the other hand, information such as a size and a shape of the light 701 generated by the light source 610 and time (light emission information) is stored in the image information generation apparatus 604 of the photoacoustic signal. In the image information generation apparatus 604 of the photoacoustic signal, the image signal of the subject 602 is generated on the basis of the ultrasonic wave reception signal 704 and the light emission information, and this image signal is output as the reproduction image information 705 based on the photoacoustic signal.

The subject 602 is displayed as an image on the basis of two pieces of information including reproduction image information based on the ultrasonic wave transmission and reception and reproduction image information based on the photoacoustic signal in the image display device 605.

Since the probe according to the present exemplary embodiment has the respectively high slice resolutions using the pulse echo technique and the photoacoustic imaging technique, it is possible to acquire image qualities in which both the slice resolutions of the pulse echo image and the photo acoustic image are excellent. In addition, since the probe can be used as both the probe using the pulse echo technique and the probe using the photoacoustic imaging technique, positional deviations for the respective images can be reduced.

### Seventh Exemplary Embodiment

the present exemplary embodiment relates to a probe having a characteristic in electronic focus in the azimuth direction (Y axis). The other configurations are the same as those of any one of the first to sixth exemplary embodiments.

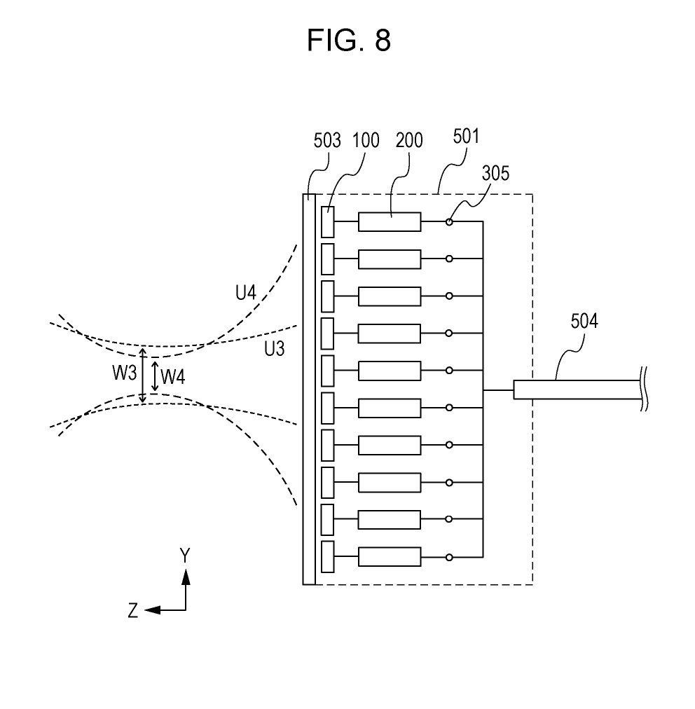

FIG. 8 is a schematic view of the search unit 502 as seen from the YZ plane. In FIG. 8, the descriptions will be given on the basis of the configuration of FIG. 5B according to the fourth exemplary embodiment. In FIG. 8, the element groups 100 in the elevation direction (the X direction) and the circuits 200 and the input/output multipurpose terminals 305 corresponding to the element groups 100 are aligned from the upper side to the lower side on paper for each column. The surface of the transmission and reception surface 503 in the Y direction does not have a curvature.

First, the electronic focus will be described. In the apparatus connected to the search unit 502, by applying a predetermined delay to the applied transmission pulse for each element group 100 in the elevation direction, the intensity distribution of the sound wave can also be applied in the azimuth direction (the Y direction). Similarly, after the reception signal is taken into the apparatus, the predetermined delay is applied to be added, so that the intensity distribution can also be applied to the detection sensitivity for the sound wave.

According to the present exemplary embodiment, settings for the electronic focus at the time of the pulse echo operation and settings for the electronic focus at the time of the photoacoustic operation are relatively changed. It is characterized in the present exemplary embodiment that the number of element groups 100 used for the electronic focus at the time of the photoacoustic operation is set to be higher than the number of element groups 100 used for the electronic focus at the time of the pulse echo operation. A width L1 of the sound wave contour lines U1 (see FIG. 2) in the elevation direction (the X direction) and a width L3 of sound wave contour lines U3 in the azimuth direction at the time of the pulse echo operation are set to be substantially equal to each other. In addition, a width L2 of the sound wave contour lines U2 (see FIG. 2) in the elevation direction (the X direction) and a width L4 of sound wave contour lines U4 in the azimuth direction at the time of the photoacoustic operation are set to be substantially equal to each other.

Accordingly, the slice resolutions in the elevation direction and the azimuth direction at the time of the pulse echo operation can be substantially matched with each other, and also the slice resolutions in the elevation direction and the azimuth direction at the time of the photoacoustic operation can be substantially matched with each other. For this reason, with the probe according to the present exemplary embodiment, since the resolutions for the pulse echo operation and the photoacoustic operation can be substantially matched with each other in the elevation direction and the azimuth direction, it is possible to provide the image in which the resolutions are substantially uniform irrespective of the directions.

According to the configuration of the exemplary embodiment of the present invention, it is possible to perform both the transmission and reception using the pulse echo technique and the reception using the photoacoustic imaging technique.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-229606, filed Nov. 12, 2014, which is hereby incorporated by reference herein in its entirety.

What is claimed is:

1. A probe comprising:
a plurality of element groups, the element group comprising a first element and a plurality of second elements which are arranged in a first direction, the plurality of element groups being arranged in a second direction intersecting with the first direction; and
an acoustic lens that is arranged on the first element and has a curvature in the first direction,
wherein the plurality of element groups is configured so that, when transmission and reception based on a method of transmitting an acoustic wave to a subject and receiving a reflected wave from a subject are performed, the transmission and reception are performed with respect to the subject by using only the first element, and when reception of a photoacoustic wave is performed, the reception from the subject is performed by using the entire element group constituted by the first element and the plurality of second elements.

2. The probe according to claim 1,
wherein the first element and the plurality of second elements are connected, for each element, to a detection circuit configured to detect a signal from the element,
the probe further comprising an adding circuit configured to perform delay and sum processing on an output signal from the detection circuit to which the first element is connected and output signals from the plurality of detection circuits to which the plurality of second elements are respectively connected, which belong to the same element group.

3. The probe according to claim 2,
wherein the output signal from the detection circuit to which the first element is connected is used at the time of the reception based on the method, and
wherein the output signal from the adding circuit is used at the time of the reception of the photoacoustic wave.

4. The probe according to claim 2, further comprising a multipurpose terminal configured to output the output signal from the detection circuit to which the first element is connected and the output signal from the adding circuit for each element group.

5. The probe according to claim 4, further comprising a terminal to which a switch switching signal is input which is used for selecting the output signal from the detection circuit to which the first element is connected or the output signal from the adding circuit, to be output from the multipurpose terminal.

6. The probe according to claim 4, further comprising a circuit configured to detect an input of a high voltage transmission signal at the time of the transmission based on the method and select the output signal from the detection circuit to which the first element is connected or the output signal from the adding circuit to be output from the multipurpose terminal on the basis of the detection result.

7. The probe according to claim 2, further comprising a terminal used as both a terminal configured to output the output signal from the detection circuit and a terminal to which a high voltage transmission signal at the time of the transmission based on the method is input for each element group.

8. The probe according to claim 2, further comprising a protection circuit configured to protect the detection circuit to which the first element is connected, from a high voltage transmission signal at the time of the transmission based on the method.

9. The probe according to claim 1, wherein at least one of the first element and the plurality of second elements has at least one cell corresponding to an electrostatic capacitance type transducer.

10. The probe according to claim 2,
wherein the first element and the plurality of second elements both have at least one cell corresponding to an electrostatic capacitance type transducer,
wherein, on a substrate on which the first element and the plurality of second elements are provided, a plurality of through holes penetrating through the substrate and a plurality of electrodes that are arranged on a back surface of the substrate in a region where the first element is arranged and are respectively connected to the plurality of through holes are provided, and
wherein each of the first element and the plurality of second elements is connected to the detection circuit via the electrode connected to the through hole.

11. The probe according to claim 1, wherein the element group to be used is changed with regard to a direction intersecting with the first direction at the time of the transmission and reception performed by using the method and the time of the reception of the photoacoustic wave in a manner that a slice resolution in the first direction and an azimuth resolution in the second direction at the time of the transmission and reception performed by using the method are substantially matched with each other, and the slice resolution and the azimuth resolution at the time of the reception of the photoacoustic wave are substantially matched with each other.

12. The probe according to claim 1, wherein the element group includes the single first element and the plurality of second elements as one group.

13. The probe according to claim 1, wherein the element group includes the first element arranged in a central part and the second elements arranged at sides of the first element in the first direction as one group.

14. The probe according to claim 1, wherein the element group includes the single first element arranged in a central part and the plurality of second elements arranged at each side of the first element in the first direction as one group.

15. The probe according to claim 1, wherein the second direction is a direction orthogonal to the first direction.

16. The probe according to claim 1, wherein a portion that is made of the same material as the acoustic lens and does not have a curvature in the first direction is arranged on the plurality of second elements.

17. The probe according to claim 1, wherein an area of a region where the acoustic lens is arranged is substantially equal to an area of a region where the acoustic lens is not arranged on a transmission and reception surface.

18. An information acquiring apparatus comprising:
be according to claim 1; and
an acquiring unit configured to acquire information of the subject by using a signal from the probe.

19. The information acquiring apparatus according to claim 18, wherein the probe transmits an acoustic wave to the subject, receives an acoustic wave reflected from the subject, and further receives an acoustic wave generated by irradiating the subject with light.

20. The information acquiring apparatus according to claim 18, further comprising a light source that irradiates the subject with light.
